(19) United States
(12) Patent Application Publication
(10) Pub. No.: US 2004/0215088 A1
(43) Pub. Date: Oct. 28, 2004

(54) Electrocardiographic signal recording with remote patient data entry

(76) Inventor: Mark Hubelbank, Sudbury, MA (US)

Correspondence Address: FISH & RICHARDSON PC, 225 FRANKLIN ST, BOSTON, MA, 02110, US

(21) Appl. No.: 10/424,679

(22) Filed: Apr. 28, 2003

(51) Int. Cl. A61B 5/0402

(52) U.S. Cl. 600/509

(57) ABSTRACT

A portable machine for recording electrocardiographic signals from a patient, the machine having a recording device for collecting electrocardiographic signals from a patient, the recording device further including a removable memory device for storing the electrocardiographic signals, a remote programming device including a remote connection with said recording device for wireless communication therewith, and patient lead wires with electrodes constructed and arranged to attach to the skin of the patient to furnish the electrocardiographic signals to said recording device.

This invention relates in general to an electrocardiographic signal recording device and more particularly apparatus and techniques for controlling the recording device remotely.

### BACKGROUND OF THE INVENTION

Conventional approaches to recording ambulatory electrocardiographs (using, for example, a Holter-type monitor system) use an analog or digital recording device connected to a number of electrodes on the patient's body. To verify correct operation the technician connecting the recorder to the patient is normally provided with some display on the recording device to assist in determining that the signal quality from the electrodes is acceptable. Once the patient is connected to the device, there is a need for the patient to indicate when specific events occur. This is commonly done with one or more event buttons on the recording device. When the patient needs to specify that a significant event has happened, it is necessary for the patient to physically access the recording device, find the appropriate button and depress it.

As these recording devices have become smaller, it has become more desirable to make the recording device as inconspicuous as possible to improve patient acceptability. The effort to reduce the size of the recording device has been limited by the need for the technician to access the recorder to initiate and verify proper operation at the beginning of the recording. It has also been limited by the need for the patient to be able to access the event button or buttons.

It is an object of the invention to eliminate the limitations caused by the need to make the recorder accessible at the start of and during the recording by allowing those functions to be performed remotely.

Another object of the invention is to achieve one or more of the preceding objects while recording all the recorded data, including the event indications on a single removable memory device to facilitate easy transfer of the recorded data to another location.

Yet another object of the invention is to achieve one or more of the preceding objects while providing identification of the recorded data in the removable memory to reduce error and facilitate tracking of data.

Still another object of the invention is to achieve one or more of the preceding objects while detecting pacemaker activity using a digital signal processing method to record the pacemaker activity in the same recorder.

One additional object of the invention is to achieve one or more of the preceding objects and provide a digital method of recovering quickly from the large overload that is imposed on the recording device by an attempt to use a defibrillator on a patient. Commonly this causes a period of ten seconds or more during which no data or only corrupted or distorted data is recorded.

### BRIEF SUMMARY OF THE INVENTION

According to the invention, a recording device includes one or more remote devices to control it, as well as a removable storage memory for recording all the electrocardiographic signals, other physiological patient data, the associated pacemaker data, the patient event indications, or the patient identification data. Specifically, it consists of one or more lead wires which are connected to electrodes on the patient. The number of these lead wires can vary according to the nature of the medical requirements. The signals from the lead wires are amplified by a series of input amplifiers. The resulting signals are then digitized. This digitization may be done in a single analog to digital converter or by multiple converters to facilitate the detection of the pacemaker signals. The digitized signals are then combined and converted into a digital format suitable for storage on a removable digital medium. A typical medium includes, for example, a compact flash device, smart media, multimedia cards, secure digital (SD) cards, or a microdrive. These are commercially available storage devices having specifications meeting industry standards.

To provide the technician with the ability to configure the recording device, enter patient identification and verify the proper operation of the device, a remote device with a display and data entry capability can be remotely connected to the recording device. This remote connection may comprise any of a number of means including, for example, radio-frequency (RF), infrared, or ultrasonic, without affecting the nature of the invention. This remote capability normally provides two-way communication with the recording device so that information may be entered on the remote device for control or storage in the recording device. Data may then be sent from the recording device to the remote device for display so that the technician may verify the data and operation of the recording device.

During actual use, the recording device may be located discretely under some portion of the patient's clothing. During this time the patient will need to, from time to time, indicate that an event has occurred, such as for example, to transmit an event marker correlating to a patient's diary entry. To allow this operation without requiring the patient to access the hidden recording device, a remote device is provided as part of this invention to send the event information to the recording device. This remote link must be a link not requiring physical contact or line of sight. Thus, the preferred mode of transmission is radio frequency although other modes such as ultrasonic and infrared are contemplated. In addition, the transmission between the remote device and the recording device may be two-directional but in its simplest form, it may be unidirectional and permit the patient to send information to the recording device, as feedback from the recording device to the patient may be desirable, but is not a requirement for all situations where this invention may be used.

Also during the recording, the amplified signal is digitized at a rate sufficiently high to detect pacemaker signals. A pacemaker signal is generally characterized by a sudden onset with a time from baseline to the peak of the signal of less than 50 microseconds. In addition, the end of the pace pulse signal also makes the transition from the peak value back to the baseline in less than 50 microseconds. Further, the peak of the pacemaker pulse generally stays at a relatively constant value for the duration of the pulse. That peak may have a typical duration of between about 500 and 2500 microseconds. The recording device of the invention determines the presence of a pace pulse and takes digital samples of the electrocardiographic signal at a high enough frequency to ensure that during the shortest valid pace pulse there will be multiple samples during a potential pace pulse. Once the digital processor detects a transition which represents a possible leading edge of a pace pulse, the processor verifies that the pace pulse remains at the approximately same amplitude until there is a final transition back to baseline. It also verifies that the final transition occurs in a sufficiently short time.

The signals acquired by the recording device have an arbitrary but irrelevant DC component. This component can significantly exceed the value of the desired signal. Instead of removing this component using a passive high-pass filter as is commonly done, this invention uses digital compensation. When the digital processor detects that the input signal is approaching one of the limits of the range of the analog-to-digital converter, it modifies the output of an additional digital-to-analog converter. This output is used to drive an offset compensation input in the input amplifiers. In this manner a large input signal offset can be compensated for in one sample period (typically a few milliseconds) rather than the 1 to 10 second period that would be required by a passive high pass-filter.

A further understanding of the nature and advantages of the invention will become apparent by reference to the remaining portions of the specification and drawings.

### BRIEF DESCRIPTION OF THE SEVERAL VIEWS OF THE DRAWINGS

FIG. 1 is a schematic view according to the invention, including its connection to the patient and the remote devices;

FIG. 2 is a block diagram of the recorder including all patient input, processing, storage and remote interface sections;

FIG. 3 is a block diagram depicting the detail implementation of each input amplifier; and

FIG. 4 is a detailed implementation of an event activation device.

### DETAILED DESCRIPTION

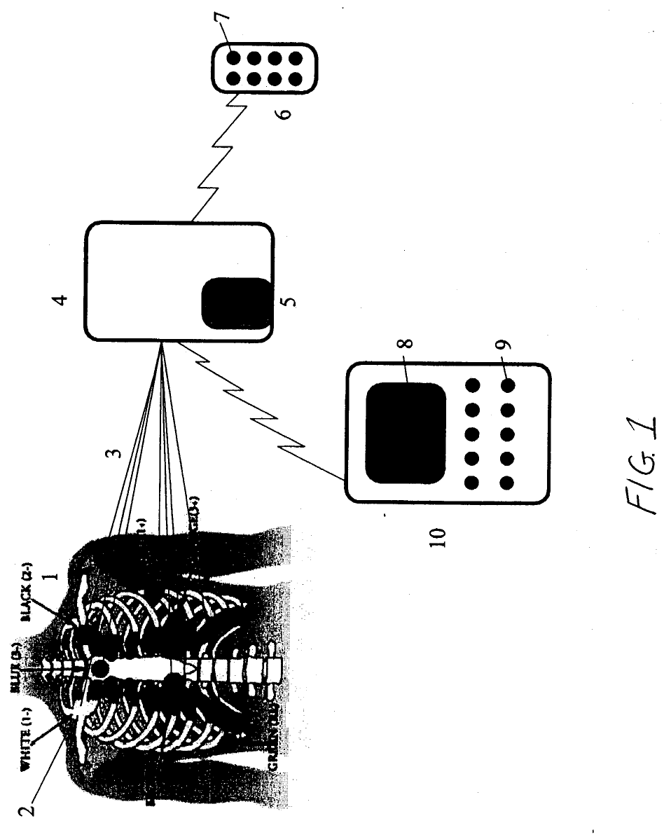

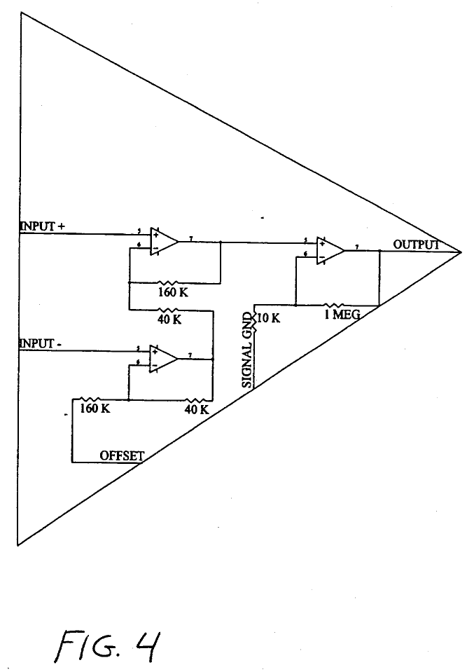

With reference to the drawings and more particularly FIG. 1, one embodiment according to the invention is shown as in use. One or more electrodes 1 are attached to the patient 2. The electrodes 1 are individually connected to the recording device 4 with lead wires 3. The recorder device 4 contains a removable memory device 5 and receivers and transmitters used to communicate with the remote programming device 10 and patient event activation device 6. One detailed example of an implementation of the event activation device 6 is depicted in FIG. 4. In typical use, after the technician has applied the electrodes 1 and connected the lead wires 3, the remote programmer 10 may be used to view the quality of the signal obtained from the electrodes 1 on the screen 8. The recording settings and patient identification can be entered using a combination of the keypad 9 and portions of the screen 8 as a touch pad.

Once the recording has been started, the recorder 4 may be hidden as desired under the patient's clothing. All further communications with the recorder is then through the event activation device 6. Pressing one or more keys 7 to indicate a specific type of event will cause a corresponding signal to be sent to the recorder 4. If so configured, the recorder 4 may transmit a confirming signal back to the specific event activation device 6. This will permit an indication to be made that the event was recorded and the time of the event. The time can then be used by the patient to mark an event diary with further information if required.

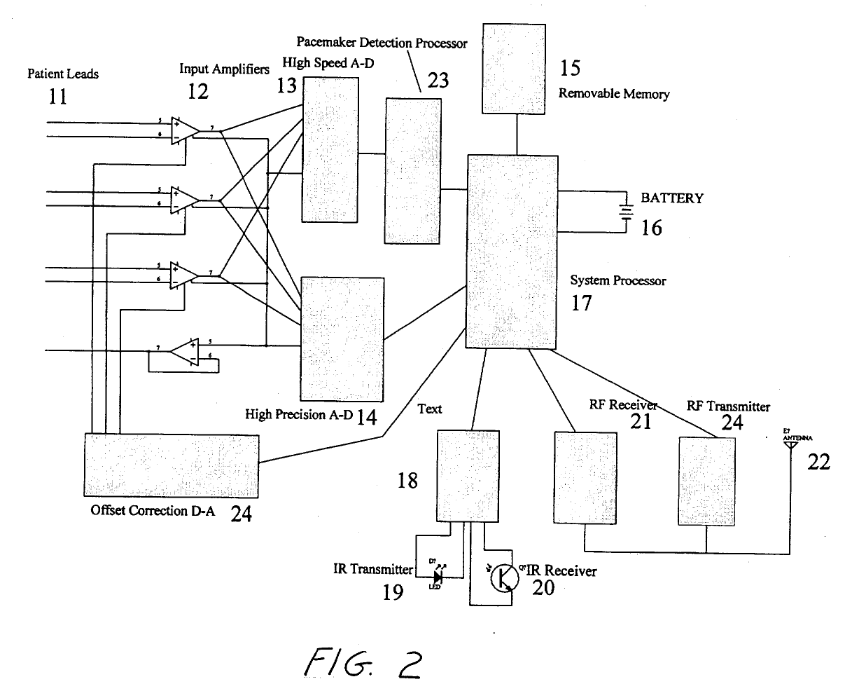

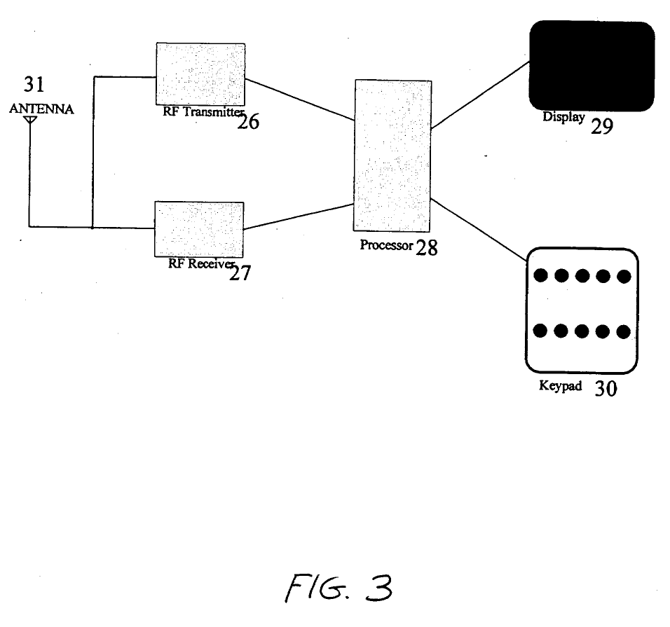

The details of the recorder 4 are further shown in FIG. 2. The electrocardiographic signals from the patient electrodes 1 are transmitted via patient leads 11 and combined with an offset correction signal from the Offset correction digital-to-analog converter D-A 24 and amplified by one or more input amplifiers 12. In a typical configuration, each input amplifier 12 will have an output which is the sum of 500 times the difference between the input signals to the amplifier and 100 times the offset correction signal. This will allow a 3-volt range offset correction D-A to compensate for a 600 millivolt total range of input signal DC offset. This is done using a typical instrumentation amplifier as shown in FIG. 3 for each of the input amplifiers.

The output of the amplifiers 12 are used as input for a high precision analog-to-digital converter A-D 14. The high precision A-D 14 digitizes the input signal with the precision necessary to represent all details of the electrocardiograph. Typically this requires a resolution of approximately 6-microvolts. After amplification, this represents a resolution of 3 millivolts. This converter will typically sample each input at a rate of at least 180 samples per second. The resulting data is passed to a system processor 17 to be formatted for storage in a removable memory device 15. If the system processor detects that the input signal is causing the input to the high precision A-D 14 to be too near its range limit, such as, for example, during the use of a defibrillator on the patient, it will send a new offset to an offset correction D-A 24 to keep the output of the input amplifiers in the input range of the high precision A-D 14. The value of the offset is added to the digital output of the high precision A-D 14 to eliminate any discontinuity that would otherwise result in the data stored in the removable memory device 15.

The amplified outputs of the input amplifiers 12 are also passed to a high speed A-D converter 13. The high-speed A-D 13 is controlled by a digital processor dedicated to the detection of pacemaker signals 23. This subsystem (the A-D 13 and processor 14) measures the signal from each amplifier 12 at a high rate, typically every 100 microseconds. If a large transition is detected in any amplifier output that meets the requirements for a pacemaker signal (typically a 2 millivolt change referred to the input in under 100 microseconds) then that channel is sampled and measured at an even higher rate of typically once every 30 microseconds. The processor uses these measurements to determine if a valid pacemaker signal has been detected. The typical criteria, which may be modified as a function of the recorder setup using the remote programmer 10, would require that all samples after the initial transition change no more than a fixed percentage between sequential samples. In one exemplary embodiment, this percentage is a reduction of 20 percent from one sample to the next. Finally, in a preferred embodiment, the end of the pulse makes a transition in under 100 microseconds.

When a valid pacemaker signal is detected by a pacemaker processor 23, the detection is signaled to the system processor 17 to be stored in the removable memory device 15 along with additional data.

At times it is desirable to have a form of operator interaction with the recording device which can not be provided by any simple buttons on its surface. To provide this capability, bidirectional communication is provided by a communications interface 18 which drives an infrared transmitter 19 and receives input from an infrared receiver 20. Using this communications port to communicate with an external device in the form of, for example, a personal digital assistant (PDA), permits a sophisticated user interface to be provided without increasing the size of the recording device 14.

During normal recording, the patient 2 may need to indicate the occurrence of events using the event activation device 6 (FIG. 1). To reduce power consumption, the RF receiver 21 may remain in a low power mode most of the time. On a low duty cycle basis, for example, between about 1 to 3 percent of the time, the receiver 21 may be turned on to check for the presence of a signal from the event activation device 6. When the patient 2 depresses a button 7 on the device 6, the transmitter 26 (FIG. 3) in the device sends an encoded signal. This signal is transmitted for a duration sufficient to ensure that the receiver will be on for at least some part of the transmitted signal. Once the system processor 17 detects that some signal is being received by the receiver 21, it locks the receiver 21 on to determine the nature of the signal. If the signal is encoded using one of the codes that corresponds to a key 7, it will record this event in the removable memory device 15. If the code does not respond to a valid key, then nothing will be recorded. In either case, after such a determination is complete, the receiver 21 will return to the low duty cycle standby.

Depending on the use to which the recording device 4 is put, it may be desired to have a message displayed on the event activation device 6. When it is desirable, for example, to verify that the event was recorded and to indicate the time of recording, the processor will activate the RF transmitter 24 and send an encoded message. This is received by the receiver in the event activation device 6. As was the case with the recorder receiver 18, the receiver 27 (FIG. 3) in the event activation device 6 may operate in a low duty cycle standby mode to save power. Thus, in one embodiment, the recorder transmitter 21 must transmit its message long enough so that the receiver in 6 will come out of standby mode and receive and decode the message. The message is then displayed on the display 25. In other embodiments, communication with the event activation device 6 is unidirectional, and accordingly, the transmitter 24 and the receiver in the event activation device 6 and its display 25 are not required.

It is evident that those skilled in the art may now make numerous uses and departures from the specific apparatus and techniques disclosed herein without departing from the inventive concept. Consequently, the invention is to be construed as embracing each and every novel feature and novel combination of features disclosed herein and limited only by the spirit and scope of the appended claims.

What is claimed is:

1. A portable machine for recording electrocardiographic signals from a patient, the machine comprising:
a recording device for collecting electrocardiographic signals from said patient, said recording device further comprising a removable memory device for storing the electrocardiographic signals;
a remote programming device including a first remote connection with said recording device for wireless communication therewith; and
patient lead wires with electrodes constructed and arranged to attach to the skin of the patient to furnish the electrocardiographic signals to said recording device.

2. The machine in accordance with claim 1 wherein said recording device is adapted for collecting pacemaker activity from said patient and said removable memory device is adapted to store said pacemaker activity.

3. The machine in accordance with claim 2 wherein said recording device comprises a digital signal processor to record the pacemaker activity together with electrocardiographic signals in the same recorder.

4. The machine in accordance with claim 2 further comprising an event activation device including a second remote connection with said recording device for wireless communication therewith, thereby permitting the patient to send event indications to the recording device.

5. The machine in accordance with claim 2 wherein said recording device is adapted for detecting collecting oxygen saturation signals from said patient.

6. The machine in accordance with claim 1 wherein the remote programming device further comprises a user display and data input device.

7. The machine in accordance with claim 1 wherein the removable memory device is a compact flash, multi-media or smart-media card.

8. The machine in accordance with claim 4 wherein the first remote connection is a radio-frequency connection.

9. The machine in accordance with claim 4 wherein the first remote connection is an ultrasonic connection.

10. The machine in accordance with claim 4 wherein the first remote connection is an infrared connection.

11. The machine in accordance with claim 10 wherein the second remote connection is a radio-frequency connection.

12. The machine in accordance with claim 10 wherein the second remote connection is an ultrasonic connection.

13. The machine in accordance with claim 10 wherein the second connection is an infrared connection.

14. The machine in accordance with claim 1 wherein the first remote connection is unidirectional.

15. The machine in accordance with claim 1 wherein the first remote connection is bi-directional.

16. A method of recording electrocardiographic signals in the removable memory device of the portable machine of claim 1 comprising:
attaching said electrodes to the skin of a patient to acquire said electrocardiographic signals and pacemaker pulses;
converting the electrocardiographic signals and pacemaker pulses into corresponding digital signals;
amplifying the digital signals;
storing the corresponding digital signals in said removable memory device of said recording device; and
sending information from said recording device to said remote programming device.

17. The method in accordance with claim 16 further comprising an event activation device including a second remote connection with said recording device for wireless communication therewith, and further comprising the step of sending event indications to the recording device.

18. The method in accordance with claim 16 further comprising the step of maintaining a continuous recording of electrocardiographic signals during the use of a defibrillator on the patient.

19. The method in accordance with claim 16 wherein said information comprises patient identification data.

20. The method in accordance with claim 16 wherein said information comprises patient event data.
